(19) United States
(12) Patent Application Publication
(10) Pub. No.: US 2006/0099235 A1
(43) Pub. Date: May 11, 2006

(54) Medical devices and compositions useful for treating or inhibiting restenosis

(75) Inventors: Adam Blakstvedt, Big Lake, MN (US); Jesus Wilfredo Casas-Bejar, Brooklyn Park, MN (US); Jamie Rae Williams, St. Louis Park, MN (US); Molly B. Schiltgen, Lake Elmo, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/987,021

(22) Filed: Nov. 11, 2004

(51) Int. Cl. A61K 48/00; A61F 13/00

(52) U.S. Cl. 424/422; 424/93.2

(57) ABSTRACT

Medical devices and related methods for making and using same suitable for treating or inhibiting restenosis are proved. Specifically, compositions and methods for I kappa B alpha (IkBα)nuclear factor kβ (NFkβ) complex breakdown inhibition are provided. One embodiment includes a CRM-1 protein binding composition such as leptomycin B. Another embodiment includes a combination of a CRM-1 protein binding composition and a nucleic acid encoding for mammalian IkBα. Medical devices disclosed include catheters and vascular stents.

### FIELD OF THE INVENTION

The present invention relates to medical devices useful for delivering anti-restenotic compositions. Specifically, the present invention provides vascular stents having controlled release coatings for delivering cytostatic compositions including anti-proliferative antibiotics, genes encoding anti-inflammatory proteins and combinations thereof, wherein the cytostatic compositions and genes encoding anti-inflammatory proteins possess anti-restenotic properties. Alternative methods for delivering the anti-restenotic compositions of the present invention are also provided.

### BACKGROUND OF THE INVENTION

Minimally invasive medical devices have proven to be useful for delivering compositions that treat medical conditions within a patient's body. Depending upon the conditions being treated, today's minimally invasive medical devices can used to deliver a bolus of a therapeutic composition and subsequently removed (for example an infusion catheter), or be implanted into the patient wherein the medical device is either adjunct to the therapy, as in the case of vascular stents, or merely serves as a drug delivery reservoir. The implantable medical device can also be used to regulate or control the therapeutic composition's release rate.

Recently, a variety of medical device coatings have been developed that control the release rate and profile of therapeutic compositions for periods of time ranging from days to years. A wide variety of such coated medical devices are useful for delivering therapeutic compositions to specific sites within the body. Examples include structural implants such as stents and vascular grafts, in-dwelling devices such as probes, catheters, shunts and sensors for monitoring, measuring and modifying biological activities within a patient, medicinal pumps, sutures, and microinjection devices including balloon catheters and needle bearing catheters. Other types of medical implants for treating different types of medical or disease conditions can include ports, valves, plates, barriers, supports, shunts, discs, and joints, to name a few.

Recently, a wide variety of medical devices and therapeutic compositions have been developed to treat cardiovascular disease, specifically, atherosclerosis-related coronary disease. One form of cardiovascular disease, commonly referred to as atherosclerosis, coronary artery diseases, which remains a leading cause of death in developed countries. Atherosclerosis is a disease that may result in the narrowing, or stenosis, of blood vessels which can lead to heart attack or stroke. Cardiovascular disease caused by stenotic or narrowed coronary arteries is commonly treated using either coronary artery by-pass graft (CABG) surgery to circumvent the blockage, or a less invasive procedure called angioplasty where a balloon catheter is inserted into the blocked coronary artery and advanced until the vascular stenosis is reached by the advancing balloon. The balloon is then inflated to deform the stenosis open, restoring blood flow.

However, angioplasty or balloon catheterization can result in internal vascular injury which may ultimately lead to reformation of narrowing vascular deposits within the renarrowing of the previously opened artery. This biological process whereby a previously opened artery becomes re-occluded is called restenosis. One angioplasty variation designed to reduce the possibility of restenosis includes the subsequent step of arterial stent deployment within the stenotic blockage opened by the expanded balloon. After arterial patency has been restored by expanding the angioplasty balloon to deform the stenotic lesion open, the balloon is deflated and a vascular stent is inserted into the tubular bore or vessel lumen across the stenosis site. The catheter is then removed from the coronary artery lumen and the deployed stent remains implanted across the opened stenosis to prevent the newly opened artery from constricting spontaneously or narrowing in response to the internal vascular injury resulting from the angioplasty procedure itself. However, restenosis following stent implantation still occurs in approximately 30% of the cases.

Treating restenosis generally requires additional, more invasive, procedures including CABG. Consequently, methods for inhibiting restenosis, or for treating incipient forms of restenosis, are being aggressively pursued. One promising method for inhibiting restenosis is the administration of medicaments that block vascular smooth muscle cell (VSMC) proliferation and migration which cause thickening of the vessel wall and subsequent narrowing of the artery. Representative anti-restenotic medicaments include cell cycle inhibitors such as anti-neoplastic agents, anti-inflammatory medicaments that block local invasion/activation of monocytes thus preventing the secretion of growth factors that may trigger VSMC proliferation and migration, and metabolic inhibitors that disrupt protein synthesis and/or intracellular transport.

Recently, significant research has been conducted utilizing compounds that inhibit cell cycle progression or completion. For convenience, the mammalian cell cycle has been divided into four discrete segments. Mitosis and cell division occur in the M phase which lasts for only about one hour. This is followed by the G₁ phase (G for Gap) and then the S phase (S for Synthesis) during which time DNA is replicated, and finally G₂ phase during which the cell prepares for mitosis. Eukaryotic cells in culture typically have cell cycle times of 16-24 hours; however, in some multicellular organisms the cell cycle can last for over 100 days. Furthermore, some cells such as neurons stop dividing completely in the mature mammal and are considered to be quiescent. This phase of the cell cycle is often referred to as G₀.

Variations in non-quiescence cell cycle times depend largely on the duration of the G₁ phase. Therefore, it is logical that a significant number of anti-proliferative cell cycle inhibitors target cellular functions occurring during G₁. However, cell cycle inhibition is not limited to agents that selectively target the G₁ phase. For example, a number of cytotoxic compounds that either inhibit mitotic spindle formation or mitotic spindle separation are known. These compounds, such as paclitaxel, target the M phase of the cell cycle. Compounds that affect DNA syntheses such as DNA topoisomerase inhibitors block cell proliferation during the G₂ and S phase.

Moreover, protein synthesis, transport and catabolism are essential for cell growth regardless which segment of the cell cycle is involved. Continual protein turnover is essential for controlling the concentrations of regulatory proteins such as enzymes and transcription factors, for abnormal protein disposal and for supplying amino acids for fresh protein synthesis. Thus metabolic inhibitors that interfere with protein turnover by blocking transcription or interfere with intracellular protein transport are particularly interesting classes of compositions.

All cell cycle inhibitors are potentially toxic when administered systemically, so the drug concentrations necessary to inhibit restenosis cannot be safely achieved using systemic administration. Consequently, in situ, site-specific drug delivery systems have been developed. Drug-eluting stents have been particularly useful because they not only provide mechanical support to maintain vessel patency, but they also release anti-restenotic agents directly into the surrounding tissue. This site specific delivery allows clinically effective drug concentrations to be achieved locally at the stenotic site without subjecting the patient to the side effects associated with systemic drug delivery. Moreover, localized or site-specific delivery of anti-restenotic drugs eliminates the need for more complex specific cell-targeting technologies intended to accomplish similar purposes.

Recent studies suggest that the anti-restenotic drug release rate and profile (collectively referred to herein after as controlled delivery) are important factors in achieving long-term restenosis prevention with minimum adverse side-effects. One method useful for the controlled delivery of anti-restenotic compositions is incorporating the compositions into a polymer used to coat the stents. Drug-eluting, polymer-coated stents using the antibiotic, immunosuppressive compound rapamycin, and paclitaxel, an anti-cancer drug that disrupts the cell cycle, have achieved some success in the clinic. However, neither composition is completely free of adverse side effects and therefore alternative methods and compositions for treating restenosis are desirable.

### SUMMARY OF THE INVENTION

The present invention provides anti-restenotic compositions, associated devices and methods useful for treating or inhibiting restenosis. One embodiment of the present invention provides novel anti-proliferatives that suppress the intracellular breakdown of the I kappa B alpha (IkBα-nuclear factor kβ (NFkβ) complex (IkBα-NFkβ). As will be explained in detail below, free (non-complex) NFkβ binds to chromosomal DNA activating a variety of inflammatory response genes including genes associated with hyperproliferation of vascular smooth muscle cells. Compositions that inhibit the intracellular breakdown of IkBα-NFkβ prevent free NFkβ from binding to chromosomal DNA and thus possess cytostatic properties useful for treating or inhibiting restenosis.

In one embodiment of the present invention the anti-proliferative compositions inhibit IkBα-NFkβ breakdown by binding to CRM-1 (chromosome region maintenance 1), a protein responsible for nuclear transport of the IkBα-NFkβ complex between the cytoplasm and nucleus. An example used in accordance with the teachings of the present invention is the CRM-1 binding compound leptomycin B.

Another embodiment of the present invention features a combination therapeutic whereby the intracellular breakdown of the IkBα-NFkβ complex is inhibited by a combination of leptomycin B and recombinant DNA encoding for IkBα (rIkBα) such that a transformed cell overexpresses IkBα in vivo assuring that intracellular NFkβ remains complexed with IkBα. The combination therapeutic of the present invention may act synergistically to further inhibit restenosis when compared to either leptomycin B or rIkBα alone. Moreover, the synergistic properties of leptomycin B ultimately reduce the need for the high transformation efficiencies traditionally associated with gene therapy techniques.

Another embodiment of the present invention provides a vascular stent having a controlled-release coating that provides anti-restenotic amounts of anti-proliferatives that suppress the intracellular breakdown of the IkBα-NFkβ complex. In one embodiment the anti-proliferative is leptomycin B and a polymer-leptomycin B combination comprises the controlled-release coating.

In yet another embodiment of the present invention, a vascular stent having a controlled-release coating providing anti-restenotic amounts of anti-proliferatives that suppress the intracellular breakdown of the IkBα-NFkβ complex wherein the anti-proliferative is a combination of a rIkBα-encoding nucleic acid vector and leptomycin B. In one embodiment the controlled release coating includes a polymer-leptomycin B combination and the rIkBα-encoding nucleic acid vector is provided using an injection catheter. In another embodiment the controlled release coating comprises a polymer-leptomycin B combination and the rIkBα-encoding nucleic acid vector.

In still another embodiment, the present invention employs a micro-syringe catheter to deliver anti-restenotic amounts of anti-proliferatives that suppress the intracellular break-down of the IkBα-NFkβ compositions peri-adventitially.

One embodiment of the present invention includes a medical device for providing the controlled release of an anti-restenotic composition comprising a vascular stent having a generally cylindrical shape comprising an outer surface, an inner surface, a first open end, a second open end and wherein at least one of said inner or said outer surfaces are adapted to provide the controlled release of an anti-restenotic effective amount of at least one I kappa B alpha (IkBα)-nuclear factor kβ (NFkβ) complex breakdown inhibitor.

In another embodiment of the present invention a vascular stent comprising a polymeric coating containing an anti-restenotic effective amount of at least one IkBα-NFkβ complex breakdown inhibitor is provided.

A further embodiment of the present invention provides a vascular stent consisting essentially of a controlled-release coating comprising a polymeric primer coat and a polymeric leptomycin-B-releasing polymer blend.

Yet another embodiment of the present invention is a method for treating or inhibiting restenosis comprising administering to a treatment site at least two IkBα-NFkβ complex breakdown inhibitors wherein a first IkBα-NFkβ complex breakdown inhibitor comprises a CRM-1 protein binding compound and a second IkBα-NFkβ complex breakdown inhibitor comprises nucleic acid encoding for mammalian IkBα.

### BRIEF DESCRIPTION OF THE DRAWINGS

FIG. 1 depicts a vascular stent having a controlled release coating made in accordance with the teachings of the present invention.

FIG. 2 depicts a vascular stent mounted on a balloon catheter ready for deployment at a treatment site in accordance with the teachings of the present invention.

FIG. 3 depicts the C-shaped configuration of an injection catheter prior to inflation suitable for use in accordance with the teachings of the present invention.

FIG. 4 depicts an inflated injection catheter and the deployed injection needle capable of penetrating the adventia

FIG. 5 depicts deployment of the compositions of the present invention directly into the adventia

FIG. 6 graphically depicts the effect of leptomycin B on human coronary artery smooth muscle cells after 3 days: Trial 1.

FIG. 7 graphically depicts the effect of leptomycin B on human coronary artery smooth muscle cells after 3 days: Trial 2.

FIG. 8 depicts a control cell culture of human coronary artery smooth muscle cells.

FIG. 9 depicts a test cell culture of human coronary artery smooth muscle cells exposed to 0.01 nM leptomycin B.

FIG. 10 depicts a test cell culture of human coronary artery smooth muscle cells exposed to 0.1 nM leptomycin B.

FIG. 11 depicts a test cell culture of human coronary artery smooth muscle cells exposed to 1 nM leptomycin B.

FIG. 12 depicts a test cell culture of human coronary artery smooth muscle cells exposed to 10 nM leptomycin B.

FIG. 13 depicts a test cell culture of human coronary artery smooth muscle cells exposed to 100 nM leptomycin B.

FIG. 14 depicts a test cell culture of human coronary artery smooth muscle cells exposed to 1000 nM leptomycin B.

FIG. 15 depicts a balloon catheter useful for deploying a stent and administering the CRM-1 binding compositions in accordance with the teaching of the present invention.

FIG. 16 depicts a balloon catheter useful for administering the CRM-1 binding compositions in accordance with the teaching of the present invention.

### DEFINITION OF TERMS USED

Anti-restenotic effective amount: As used herein “anti-restenotic effective amount” refers to an amount of a composition that suppresses the intracellular breakdown of the IkBα-NFkβ complexes, as defined below, sufficient to reduce in-stent restenosis as compared with restenosis rates in patients undergoing PTCA and stent deployment alone.

Controlled release: As used herein “controlled release” refers to the release of an anti-restenotic compound from a medical device surface at a predetermined rate. Controlled release implies that the anti-restenotic compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of anti-restenotic composition may be desirable (for example, in the present invention an immediate “burst” of an IkBα-expressing vector may be desirable followed by a delayed release of Leptomycin B).

The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time. Examples of controlled release means include polymer coatings comprising an anti-restenotic composition-polymer mixture, a polymer barrier, or cap coat over an anti-restenotic composition coating, reservoirs formed in the stent surface that concentrate and delay anti-restenotic composition release and the like.

IkBα-NFkβ complex breakdown inhibitor: As used herein an “IkBα-NFkβ complex breakdown inhibitor” shall mean any composition that suppresses NFkβ transcription-related activity by reducing intracellular concentrations of uncomplexed NFkα. Non-limiting examples of the present invention include compositions specifically binding CRM-1 (e.g. leptomycin B), in another embodiment a target cell causes the cell to over-express IkBα thus shifting the intracellular equilibrium to favor complexed NFkβ (e.g. intracellularily expressed recombinant IkBα binding free NFkβ).

Treatment site: As used herein a “treatment site” is defined as an anatomical site within a mammalian body susceptible to restenosis. In one embodiment of the present invention the treatment site is a vessel lumen that has been previously, or simultaneously with administration of the present invention, undergone PTCA with, or without, stent deployment.

Vector: As used herein “vector” is defined as the DNA of any transmissible agent (e.g. plasmid [including naked DNA] or virus) into which a segment of foreign DNA (in the present case DNA encoding for mammalian rIkBα) can be spliced in order to introduce the foreign DNA into cells of a host and promote its replication and transmission therein.

### DETAILED DESCRIPTION OF THE INVENTION

The present invention provides compositions and related methods for treating and inhibiting restenosis following percutaneous procedures used to restore patency to blocked blood vessels. As a specific, non-limiting example, the present invention shall be described as it relates to minimally invasive, percutaneous methods used to restore patency to the coronary vasculature, specifically, percutaneous transluminal angioplasty (PTCA).

The heart receives oxygen and nutrients via blood flowing through the coronary arteries. Fatty deposits can form on the arterial wall in a process called atherosclerosis. If these deposits block off enough of the blood flow, then the patient will experience pain when exercising or at rest, known as angina. If the blockage becomes severe, then the downstream heart tissue can begin to starve and die. Physicians use several treatments for atherosclerosis. In early stages, the patient may be placed on cholesterol-lowering medications. If the blockage is serious and localized, then the interventional cardiologist may perform PTCA to open the artery with catheters, guidewires, balloons, and stents. In the worst cases, the patient can be referred to a cardiothoracic surgeon for a coronary artery bypass procedure (CABG), which uses artery or vein grafts to circumvent the blockage.

With PTCA, the interventionalist treats local sites of atherosclerosis in the arteries with devices threaded through the blood vessels. First, a major vessel, such as the femoral artery, is accessed through the groin and an introducer is inserted to form an access gateway. Next, the interventionalist passes a guidewire through the introducer and into the heart past the blockage site. Once the guidewire is in place, an angioplasty balloon catheter is run over the guidewire to the lesion site, inflated, and the coronary stent is deployed to alleviate the blockage and assure that the artery remains open.

Endothelial cell injury, resulting from balloon angioplasty or other interventional vascular procedures, can trigger cellular events leading to thrombogenic, inflammatory, and ultimately to hyperproliferative responses. As a result of the injury, smooth muscle cell migration and hyperproliferation can take place into the subintimal region of the artery, a phenomenon that has been implicated in the genesis of coronary restenosis. These processes are further complicated when the subjacent arterial disease is considered. It has been demonstrated that atherosclerosis-associated tissues have poor endothelial homeostasis and ongoing chronic Inflammation. It is very likely that in a vascular lesion, where there is already latent cell activation and abnormal physiology, minor stimuli from vascular procedures may trigger excessive responses and lead to restenosis.

In-stent restenosis occurs in about 30% of stenting procedures. Strategies that have been used to decrease the rate of restenosis include improving the stent design (changing the configuration of the metal struts to more evenly distribute load), manipulating the metal material composing the stent (using new alloys), and combining the stents with agents such as drugs or genes that can attenuate the injury response to stent implantation. One method used to deliver anti-restenosis compounds is to incorporate them into a polymer, and then coat the mixture onto stents. Anti-restenosis gene therapy is another strategy that has been explored with some level of success in the scientific community. Of particular interest is an anti-inflammatory gene therapy approach described by Breuss, et al. (Circulation. 2002; 105: 633-638). Breuss et al. report that adenoviral delivery of I kappa B alpha (IkBα) to balloon-injured arteries could attenuate lumen narrowing in atherosclerotic rabbits. I kappa B alpha is an inhibitor of nuclear factor kβ (NFkβ), a transcription factor involved in inflammation. It is well documented that inflammation is a key mechanism in the progression of atherosclerosis and in the development of restenotic lesions after angioplasty and stenting (Okamoto, et al. Circulation. 2001; 104: 2228-2235; Wilson, et al. Atherosclerosis. 2002; 160: 147-153). Nuclear factor kβ is a transcription factor that normally exists bound to IkBα in the quiescent state in the cytosol of cells. When inflammatory signals reach cells, they cause IkBα to be degraded, allowing for NFkβ to be activated. Activated NFkβ then travels into the nucleus of the cell where it binds to the genomic DNA and activates the transcription of many genes, including more inflammatory mediators. Proteins that result from NFkβ activation cause inflammation to progress and can make the atherosclerotic lesion more severe. The Breuss et al. studies demonstrated that this reaction and the resulting side effects (lumen narrowing) are attenuated when injured vessels are bombarded with extra IkBα, which keeps NFkβ bound and quiescent. This same group also showed a positive effect of IkBα gene therapy against in-stent restenosis (Cejna, et al. Radiology. 2002; 223(3): 702-708). Briefly, an adenovirus-carrying IkBα was delivered to the iliac arteries of atherosclerotic rabbits through a weeping balloon catheter immediately after nitinol stents were deployed. Four weeks after implant, a 53% reduction in neointimal formation was observed in the IkBα-treated arteries when compared with controls.

Thus, it has been demonstrated that suppression of NFkβ-induced inflammatory gene activation inhibits restenosis. Towards that end the present inventors have conceived of several different approaches to providing compositions that selectively suppress the intracellular degradation of the IkBα-NFkβ complex thus inhibiting NFkβ-induced inflammatory gene activation and restenosis.

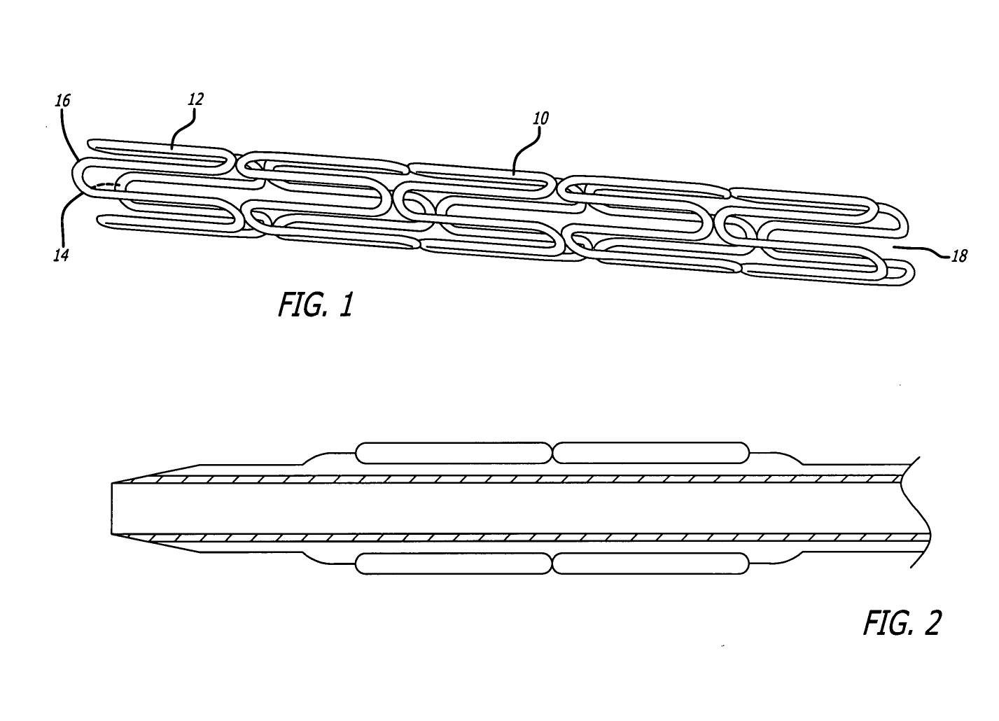

The present invention provides two unique strategies for suppressing intracellular degradation of the IkBα-NFkβ complex. In one embodiment of the present invention, the anti-inflammatory antibiotic leptomycin B is administered locally from a stent having a controlled-release coating. In another embodiment, the present invention provides a combination therapeutic whereby leptomycin B is administered locally from a stent in combination with the concomitant administration of exogenous nucleic acid encoding for recombinant IkBα (rIkBα). The exogenous nucleic acid encoding for rIkBα can be incorporated into the stent coating or administered using an injection catheter. The exogenous nucleic acid encoding for rIkBα alone or in combination with leptomycin B can be administered by injection catheter either immediately before or after stent placement to either the vessel's intimal lining or peri-adventitially using a specialized microinjection catheter such as, but not limited to, the catheter disclosed in U.S. Pat. No. 6,547,803 filed Sep. 20, 2001, the entire contents of which are incorporated herein by reference, specifically see FIGS. 1A-1C and column 4, line 30 through column 7 line 47.

Leptomycin B (also known as Elactocin) is a secondary metabolite of Streptomyces sp. that was discovered in the early 1980's (see Hamamoto T, et al. 1986. Leptomycin A & B, new antifungal antibiotics. J. Antibiotics 36: 646-650). It is known chemically as 2,10,12,16,18-nonadecapentaenoic acid; it molecular weight is 540.73 and is CAS registry number is 87081-35-4. Leptomycin B is available commercially from Synexa Life Sciences, Cape Town, South Africa. The chemical formula for leptomycin B is C₃₃H₄₆O₆ and has the structure depicted in Formula 1 below.

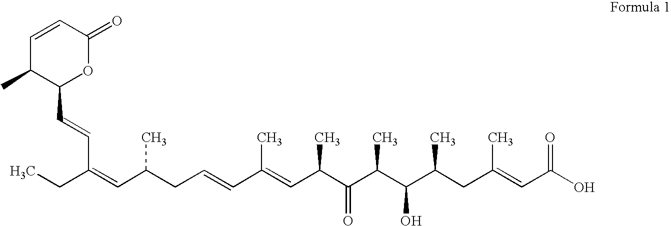

### Leptomycin B

Leptomycin B's mechanism of action has been defined and has been published in the scientific literature (Rodriguez, et al. J Biol Chem. 1999; 274(13): 9108-9115; Turpin, et al. J Biol Chem, 1999; 274(10): 6804-6812; Tam, et al. Molecular and Cellular Biology. 2000; 20(6): 2269-2284; Shrikesh et al. Molecular and Cellular Biology. 2000; 20(5): 1571-1582). Briefly, NFkβ and IkBα are found as an intracellular complex rendering NFkβ biologically inactive. The IkBα-NFkβ complex is constantly shuttled between the nucleus and the cytoplasm, but the rate of nuclear export vastly exceeds the rate of nuclear import, causing the complex to primarily reside in the cytoplasm. Nuclear export is facilitated by a nuclear membrane bound protein called CRM-1 (chromosomal regional maintenance 1). When inflammatory signals (such as TNF-α, IL-1β, or other cytokines that are typically found in an atherosclerotic lesion) are sensed by the cell, they activate the Ikβ kinase (IKK) complex. Ikβ kinase degrades IkBα, which frees NFkβ. Free NFkβ enters the nucleus, binds the genomic DNA, and activates expression of more inflammatory genes. This cascade causes the pathology to progress. As part of a negative feedback loop, NFkβ itself will activate the production of more IkBα. The newly synthesized IkBα can enter the nucleus, complex with the NFkβ and pull it off of the genomic DNA, and export NFkβ back to the cytoplasm where it can rest or be recycled. However, if the inflammatory stimulus is still there (as in an atherosclerotic lesion), the IkBα will again be degraded from the IkBα-NFkβ complex, and the inflammatory cycle will continue.

If these newly-formed IkBα-NFkβ complexes were bound in the nucleus and could not be exported to the cytoplasm, the IkBα would not be vulnerable to degradation. In that case, the IkBα would remain bound to the NFkβ, and the NFkβ would be inactive. As mentioned above, the IkBα-NFkβ complexes are shuttled from the nucleus to the cytoplasm by CRM-1. Therefore, if CRM-1 was “turned off,” the complexes would remain in the cell nucleus. This can be accomplished with leptomycin B, since its function is to bind CRM-1 (Fukuda, et al. Nature. 1997; 390: 308-311; Kudo, et al. Proc. Natl. Acad. Sci. USA. 1999; 96:9112-9117). Through this binding, leptomycin B can cause IkBα-NFkβ complexes to be “stuck” in the nucleus, preventing NFkβ from doing more damage.

In addition to being an excellent anti-restenosis candidate on its own, in another embodiment of the present invention leptomycin B is used synergistically with IkBα gene therapy, and delivered together to provide a more profound therapeutic effect than when either one is delivered alone. It has been shown that the adenoviral delivery of IkBα to injured arteries immediately after stent implantation in atherosclerotic rabbits attenuates neointimal formation (Cejna, et al. Radiology. 2002; 223(3): 702-708). Adenovirus vectors are extremely efficient at facilitating entry of therapeutic genes into cells; however, in some patients adenovirus vectors are known to induce an inflammatory response. Therefore, it may be desirable to use other vectors less prone to induce an inflammatory response. Unfortunately, these alternative vectors may be less efficient that adenovirus vectors at producing anti-restenotic effective levels of rIkBα. However, a leptomycin B/IkBα combination may overcome this problem because the leptomycin B may protect the IkBα from normal degradation mechanisms present in the cell. Thus, the lower level of IkBα protein produced by an inefficient transduction process may still provide an anti-restenotic effective amount of rIkBα.

Moreover, most gene therapy techniques have had significant difficulty in overcoming cell targeting as well as low transfection efficiencies. Low transfection efficacies has accounted for a number of failures in otherwise promising gene therapy applications. Moreover, cell targeting is a common problem associated with most forms of gene therapy and most probably contributes to low transfection efficiencies. The present invention solves these problems in two ways. First, the site-specific delivery of vectors having nucleic acids encoding IkBa are directly administered to the target site using a deployed stent or injection catheter. Thus high concentrations of competent vectors can be administered to the cell of interest without having the vector diluted by systemic administration or mixed cell populations where a minority of the cells present represent cells of interest. Secondly, because the rIkBα will act synergistically with leptomycin B to inhibit intracellular degradation of the IkBα-NFkβ complexes, high transfection efficacies are not necessarily required to provide an anti-restenotic effective amount IkBα-expressing nucleic acid.

The medical devices used in accordance with the teachings of the present invention may be permanent medical implants, temporary implants, or removable devices. For examples, and not intended as a limitation, the medical devices of the present invention may include, stents, catheters, micro-particles, probes and vascular grafts.

In one embodiment of the present invention, stents are used as the drug delivery platform. The stents may be vascular stents, urethral stents, binary stents, or stents intended for use in other ducts and organ lumens. Vascular stents may be used in peripheral, neurological or coronary applications. The stents may be rigid expandable stents or pliable self-expanding stents. Any biocompatible material may be used to fabricate the stents of the present invention including, without limitation, metals or polymers. The stents of the present invention may also be bioresorbable.

In one embodiment of the present invention metallic vascular stents (FIG. 1.) are coated with one or more anti-restenotic compound, specifically at least one CRM-1 binding compound, more specifically the CRM-1 binding compound is leptomycin B. The CRM-1 binding compound of the present invention may be dissolved or suspended in any carrier compound that provides a stable composition that does not react adversely with the device to be coated or inactivate leptomycin B. The metallic stent is provided with a biologically active leptomycin B coating using any technique known to those skilled in the art of medical device manufacturing. Suitable non-limiting examples include impregnation, spraying, brushing, dipping and rolling. After the leptomycin B solution is applied to the stent, it is dried leaving behind a stable leptomycin B delivering medical device. Drying techniques include, but are not limited to, heated forced air, cooled forced air, vacuum drying or static evaporation. Moreover, the medical device, specifically a metallic vascular stent, can be fabricated having grooves or wells in its surface that serve as receptacles or reservoirs for the CRM-1 binding compositions of the present invention.

The anti-restenotic effective amount of leptomycin B used in accordance with the teachings of the present invention can be determined by a titration process. Titration is accomplished by preparing a series of stent sets. Each stent set will be coated, or contain different dosages of leptomycin B. The highest concentration used will be partially based on the known toxicology of the compound. The maximum amount of drug delivered by the stents made in accordance with the teaching of the present invention will fall below known toxic levels. Each stent set will be tested in vivo using the preferred animal model as described in Example 2 below. The dosage selected for further studies will be the minimum dose required to achieve the desired clinical outcome. In the case of the present invention, the desired clinical outcome is defined as the inhibition of vascular re-occlusion, or restenosis below the level seen in patients receiving stents having no anti-restenotic coating. Generally, and not intended as a limitation, an anti-restenotic effective amount of the leptomycin B of the present invention will range between about 0.5 ng to 1.0 mg depending on the delivery platform selected.

Moreover, treatment efficacy may also be affected by factors including dosage, route of delivery and the extent of the disease process (treatment area). An effective amount of leptomycin B can be ascertained using methods known to those having ordinary skill in the art of medicinal chemistry and pharmacology. First the toxicological profile for leptomycin B is established using standard laboratory methods. For example, leptomycin B is tested at various concentration in vitro using cell culture systems in order to determine cytotoxicity (see Example 1 below). Once a non-toxic, or minimally toxic, concentration range is established, leptomycin B is tested throughout that range in vivo using a suitable animal model. After establishing the in vitro and in vivo toxicological profile for leptomycin B, it is tested in vitro to ascertain if the compound retains anti-proliferative activity at the non-toxic, or minimally toxic ranges established.

Finally, leptomycin B-coated stents are administered to treatment areas in humans in accordance with either approved Food and Drug Administration (FDA) clinical trial protocols, or protocol approved by Institutional Review Boards (IRB) having authority to recommend and approve human clinical trials for minimally invasive procedures. Treatment areas are selected using angiographic techniques or other suitable methods known to those having ordinary skill in the art of intervention cardiology. Leptomycin B-coated stents having a range of suitable dosages are then deployed to the selected treatment areas. Preferably, the optimum dosages will be the highest non-toxic, or minimally toxic concentration established for leptomycin B. Clinical follow-up will be conducted as required to monitor treatment efficacy and in vivo toxicity. Such intervals will be determined based on the clinical experience of the skilled practitioner and/or those established in the clinical trial protocols in collaboration with the investigator and the FDA or IRB supervising the study.

Leptomycin B therapy of the present invention can be administered directly to the treatment area using any number of techniques and/or medical devices. In one embodiment of the present invention, the leptomycin B composition is applied to a vascular stent. The vascular stent can be of any composition or design. For example, the stent may be self-expanding or a mechanically expanded stent 10 using a balloon catheter FIG. 2. The stent 10 may be made from stainless steel, titanium alloys, nickel alloys or biocompatible polymers. Furthermore, the stent 10 may be polymeric or a metallic stent coated with at least one polymer. In other embodiments the delivery device is an aneurysm shield, a vascular graft or surgical patch. In yet other embodiments, the leptomycin B therapy of the present invention is delivered using a porous or “weeping” catheter to deliver a leptomycin B-containing hydrogel composition to the treatment area. Still other embodiments include microparticles delivered using a catheter or other intravascular or transmyocardial device.

In another embodiment, an injection catheter can be used to deliver the leptomycin B either directly into, or adjacent to, a vascular occlusion or a vasculature site at risk for developing restenosis (treatment area). As used herein, adjacent means a point in the vasculature either distal to, or proximal from a treatment area that is sufficiently close enough for the anti-restenotic composition to reach the treatment area at therapeutic levels. A vascular site at risk for developing restenosis is defined as a treatment area where a procedure is conducted that may potentially damage the luminal lining. Non-limiting examples of procedures that increase the risk of developing restenosis include angioplasty, stent deployment, vascular grafts, ablation therapy, and brachytherapy.

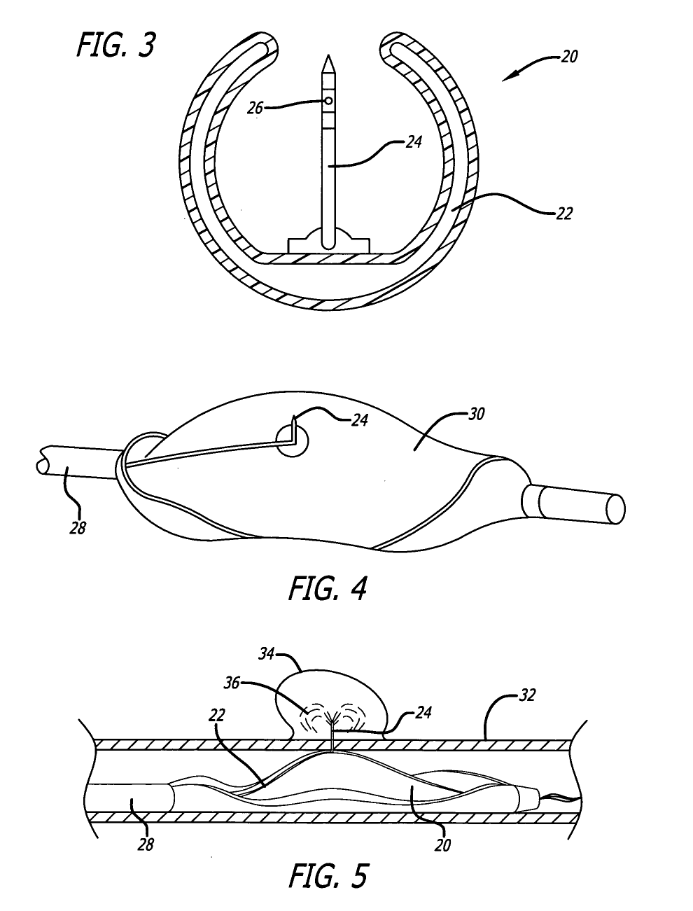

In one embodiment of the present invention an injection catheter as depicted in U.S. Pat. No. 6,547,803 can be used to administer leptomycin B directly to the adventia. FIGS. 3, 4 and 5 depict one such embodiment. FIG. 3 illustrates the C-shaped configuration of the catheter balloon 20 prior to inflation having the injection needle 24 nested therein and a balloon interior 22 connected to an inflation source (not shown) which permits the catheter body to be expanded as shown in FIG. 4. Needle 24 has an injection port 26 that transits the leptomycin B into the adventia from a proximal reservoir (not shown) located outside the patient.

FIG. 4 illustrates the inflated balloon 30 attached to the catheter body 28 and injection needle 24 capable of penetrating the adventia. FIG. 5 depicts deployment of the leptomycin B of the present invention directly into the adventia 34. The injection needle 24 penetrates the blood vessel wall 32 as balloon 20 is inflated and injects the leptomycin B 36 into the tissue.

The medical device can be made of virtually any biocompatible material having physical properties suitable for the design. For example, tantalum, stainless steel and nitinol have been proven suitable for many medical devices and could be used in the present invention. Also, medical devices made with biostable or bioabsorbable polymers can be used in accordance with the teachings of the present invention. Although the medical device surface should be clean and free from contaminants that may be introduced during manufacturing, the medical device surface requires no particular surface treatment in order to retain the coating applied in the present invention. Both surfaces (inner 14 and outer 12 of stent 10, or top and bottom depending on the medical devices' configuration) of the medical device may be provided with the coating according to the present invention.

There are many different techniques and configurations useful for providing a medical device with leptomycin B eluting surfaces. One embodiment includes a bare metal stent that has been cleaned prior to being provided with leptomycin B. The leptomycin B can be diluted in a pharmaceutically acceptable carrier including sugars, proteins and the like, or merely diluted in a suitable solvent and then applied to the stent using any appropriate technique such as, but not limited to, spray drying, rolling or dipping. In this embodiment the sent is coated with a polymer-free leptomycin coating. Alternate embodiments according to the present invention include first preparing a solution which includes a solvent, a polymer dissolved in the solvent and a leptomycin B composition dispersed in the solvent. The solvent, polymer and therapeutic substance should be mutually compatible and the solvent should be capable of placing the polymer and drug into solution at a desired concentration. It is also essential that the solvent and polymer chosen do not chemically alter leptomycin B's therapeutic character. However, leptomycin B only needs to be dispersed throughout the solvent so that it may be either in a true solution with the solvent or dispersed in fine particles in the solvent. The solution is applied to the medical device and the solvent is allowed to evaporate leaving a coating on the medical device comprising the polymer(s) and the leptomycin B composition.

Typically, the solution can be applied to the medical device by either spraying the solution onto the medical device or immersing the medical device in the solution. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the medical device. In either a coating applied by spraying or by immersion, multiple application steps are generally desirable to provide improved coating uniformity and improved control over the amount of leptomycin B composition to be applied to the medical device. The total thickness of the polymeric coating will range from approximately 1 micron to about 20 microns or greater. In one embodiment of the present invention leptomycin B is contained within a base coat, and a top coat is applied over the leptomycin B containing base coat to control release of leptomycin B into the tissue.

The polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer-to-leptomycin B composition ratio will depend on the efficacy of the polymer in securing the leptomycin B composition onto the medical device and the rate at which the coating is to release the leptomycin B composition to the tissue of the blood vessel. More polymer may be needed if it has relatively poor efficacy in retaining the leptomycin B composition on the medical device and more polymer may be needed in order to provide an elution matrix that limits the elution of a very soluble leptomycin B composition. A wide ratio of therapeutic substance-to-polymer could therefore be appropriate and could range from about 0.001 % to 99% by weight of therapeutic substance-to-polymer.

In one embodiment of the present invention, a vascular stent as depicted in FIG. 1 is coated with leptomycin B using a two-layer biologically stable polymeric matrix comprising a primer coat and a drug-eluting layer layer. Stent 10 has a generally cylindrical shape and an outer surface 12, an inner surface 14, a first open end 16, a second open end 18 and wherein the outer and inner surfaces 12 and 14 are adapted to deliver an anti-restenotic effective amount of at least one CRM-1 binding compound in accordance with the teachings of the present invention, preferably leptomycin B. Briefly, a polymer primer layer comprising parylene C or a derivative thereof is applied to stent 10 such that the outer surface 12 is coated with polymer. In another embodiment both the inner surface 14 and outer surface 12 of stent 10 are provided with polymer primer coats.

Next, an outer layer comprising leptomycin B and a drug-eluting polymer is applied to the stent's 10 outer layer 14 that has been previous provide with primer coat. In one embodiment of the present invention the drug-eluting polymer is made in accordance with the teachings of co-pending Patent Cooperation Treaty (PCT) application number PCT/US04/26516 filed Aug. 12, 2004 and incorporated herein by reference in its entirety. In another embodiment of the present invention, the drug-eluting polymer comprises a terpolymer-copolymer-homopolymer blend having from approximately 60% to 70% terpolymer, 20% to 25% copolymer and 5% to 15% homopolymer. In one embodiment of the present invention the terpolymer comprises from 70% to 80% hexyl methacrylate, 1% to 10% vinyl acetate and 15% to 20% polyvinylpyrrolidone (PVP); the copolymer comprises from approximately 90% to 99% butyl methacrylate and from 1% to 10% vinyl acetate; and the homopolymer is PVP (this terpolymer drug-eluting coating will be referred to herein after as “Matrix” for ease of reference).

In a preferred embodiment of the present invention, Matrix comprises approximately 67% of a terpolymer having 77% hexyl methacrylate, 5% vinyl acetate and 18% PVP; approximately 23% of a copolymer comprising 95% butyl methacrylate and 5% vinyl acetate and approximately 10% of the homopolymer PVP. Matrix properties control the rate at which leptomycin B elutes from the stent. The Matrix properties that most significantly effect elution rate include the polymer's glass transition point (Tg), the solubility of the leptomycin B in Matrix, and the thickness of the Matrix coating. Furthermore, the elution rate of the present invention can be tuned by changing the relative percentages of the polymers and the polymer's monomeric subunits. Additional fine tuning of elution rate can be achieved by applying a polymer cap coat, or diffusion barrier over Matrix. Suitable, non-limiting examples of diffusion barriers include biocompatible polymers such as ethylene-co-vinyl actate (EVA) and poly(butyl)methacrylate (PMB).

The Matrix/leptomycin B solution may be incorporated into or onto a medical device in a number of ways. In one embodiment of the present invention the Matrix/leptomycin B solution is sprayed onto the stent 10 and then allowed to dry. In another embodiment, the Matrix/leptomycin B solution may be electrically charged to one polarity and the stent 10 electrically changed to the opposite polarity. In this manner, the Matrix/leptomycin B solution and stent will be attracted to one another thus reducing waste and providing more control over the coating thickness. Moreover, leptomycin B can be formulated as a component of a multi-drug system designed to prevent vascular pathology such as in-stent restenosis. Representative anti-restenotics that can be co-administered with leptomycin B include, but are not limited to anti-proliferatives, immunosuppressives, anti-thrombotics, antibiotics, anti-coagulants, anti-inflammatories, and pro-healing agents. These agents can be combined with a polymer and applied as coatings to stents or grafts.

In another embodiment of the present invention, the polymer coating is bioresorbable. The bioresorbable polymer-leptomycin B blends of the present invention can be designed such that the polymer absorption rate controls drug release. In one embodiment of the present invention a polycaprolactone-leptomycin B blend is prepared. A stent 10 is then stably coated with the polycaprolactone-leptomycin B blend wherein the stent coating has a thickness of between approximately 0.1 μm to approximately 100 μm. The polymer coating thickness determines the total amount of leptomycin B delivered and the polymers absorption rate determines the administration rate.

Another embodiment of the present invention features the synergistic administration of leptomycin B and a vector having a nucleic acid encoding for mammalian recombinant IkBα (rIkBα vector). As briefly discussed above, the rIkBα vector of the present invention may be administered in combination with leptomycin B either by deploying it from the same stent coating as describe in detail above for leptomycin B alone, or separately using either a weeping catheter or injection catheter (as also described above for leptomycin B alone). However, regardless of whether the rIkBα vector is released from Matrix or a similar coating in combination with leptomycin B, or separately from a catheter, the present inventors envision that the two anti-restenotics of the present invent (rIkBα and a CRM-1 binding composition) are administered in a fashion that is compatible with the synergistic intracellular suppression of IkBα-NFkβ complex degradation.

In one embodiment of the present invention, the vector comprising a DNA sequence encoding for mammalian recombinant IkBα is a replication-defective virus selected from the group consisting of adenoviruses, retroviruses, lentiviruses, alphavirues, and herprsviruses. In another embodiment of the present invention the vector is a non-viral gene delivery system including naked DNA and liposomes. The naked DNA plasmid comprises an origin of replication, a promoter sequence, and a nucleic acid sequence encoding for mammalian rIkBα. The naked DNA plasmid according to the present invention may be co-administered with transfection-facilitating compositions such as proteins and calcium phosphate. Other non-viral vectors of the present invention include nucleic acid plasmids encoding for mammalian rIkBα surrounded by artificial lipid layers to form a lipid sphere (liposomes). In one embodiment of the present invention, nucleic acid sequences encoding for mammalian IkBα encodes for human IkBα.

A non-limiting representative embodiment of an IkBα viral vector made in accordance with the teachings of the present invention is provided in Cejna et al. (Radiology, 2002, 223:702-8). Specifically, these authors describe the replication-defective recombinant adenoviral vector rAdCMV.IkBα based on human adenovirus type 5 serotype containing a coding sequence for IkBα as further described by C J Wrighton (Wrighton C J, et al. 1996. Inhibition of endothelial cell activation by adenovirus-mediated expression of I kappa B alpha, an inhibitor of the transcription factor NF-kappa B. J. Exp. Med. 183:1013-1022, which is incorporated herein in its entirety).

The replication defective viral vectors used in accordance with teaching of the present invention were prepared using techniques known to those having ordinary skill in the art of molecular biology. Briefly, the coding sequence for IkBα used in the rAdCMV.IkBα recombinant adenoviral vector of the present invention is under the control of a cytomegalovirus (CMV) promoter. The first adenosine-thymine-guanine codon of the IkBα complementary DNA was replaced with a Bacillus amyloly type II restriction endonuclease (BamH1) restriction site by using polymerase chain reaction. Double-stranded oligonucleotide encoding an initiator methionine followed by the SV40 large T antigen nuclear localization signal and three glycine residues as a flexible spacer was ligated into the newly generated BamH1 site. The construct was sequenced to exclude possible errors generated during the amplification procedure, ligated into the vector pACCMVpLpASR+, and co-transfected with pJM17, a plasmid containing the adenoviral geneome with a deletion in the E1 region, into 293 cells by using LipotectaminPlus (Gibco-BRL; Invitrogen, Carlsbad, Calif.). Clones obtained after subcloning 293 cells were tested for IkBa expression with Western blotting by using an anti-MAD-3 antibody (Santa Cruz Biotechnology, Santa Cruz, Calif.) at a dilution of 1:1500. The recombinant adenovirus was purified using cesium chloride centrifugation.

The resulting IkBα expressing vector is then suspended in a pharmaceutically acceptable diluent such as normal saline or phosphate buffered saline to form a vector suspension. The vector suspension can thereafter be used in further pharmaceutical compounding, such as incorporated into a biocompatible polymer used to coat a vascular stent as described in detail above.

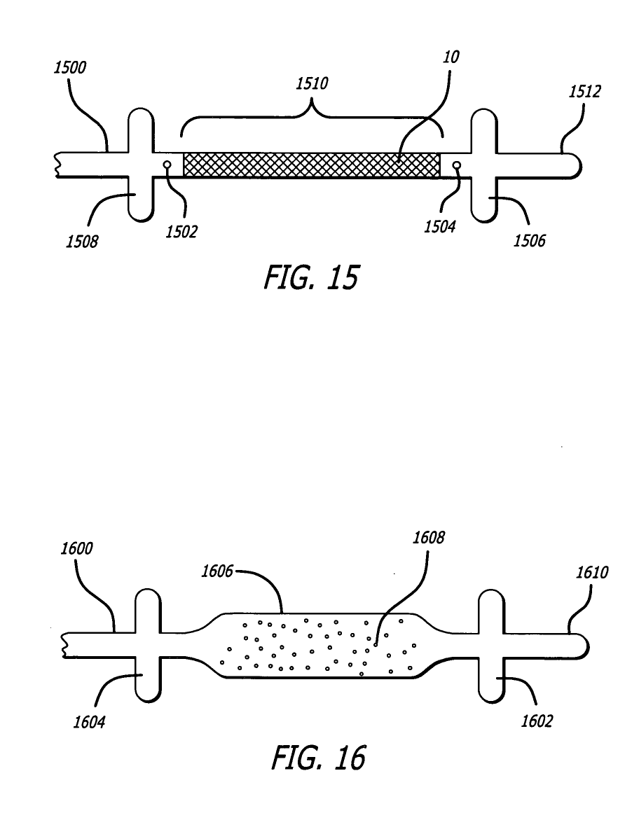

Moreover, the vector suspension can also be formulated into a hydrogel matrix and used with a weeping catheter for direct administration to the vessel wall. Briefly, in one embodiment the present invention, a balloon catheter 1500 as depicted in FIG. 15 is used to administer an IkBα expressing vector directly to the luminal wall. In this embodiment, a catheter 1500 has three balloons, distal, medial and proximal, on the distal end of the catheter tip 1512. Catheter 1500 is advanced until juxtaposed to the treatment site. Distal balloon 1506 is inflated to temporarily stop blood flow though the treatment area. Immediately after proximal balloon 1508 is inflated, a IkBα expressing vector suspension is then injected onto the luminal wall though injection port 1502 or 1504 after which a vascular stent 100, having been pre-compressed onto medial balloon 1510, is deployed by inflating medial balloon 1510. Next the distal balloon 1506, proximal balloon 1508 and medial balloon 1510 are immediately deflated and the catheter 1500 is removed leaving a CMR-1 binding composition-eluting stent in place.

In an alternative embodiment depicted in FIG. 16, a balloon catheter 1600 is used to administer an IkBα expressing vector directly to the luminal wall. In this embodiment, a catheter 1600 has three balloons, distal 1602, medial 1606 and proximal 1604, on the distal end of the catheter tip 1610. Catheter 1600 is advanced until juxtaposed to the treatment site. Distal balloon 1602 is inflated to temporarily stop blood flow though the treatment area. Immediately after proximal balloon 1604 is inflated followed by inflating medial balloon 1606, an IkBα expressing vector suspension is then injected onto the luminal wall though injection port 1608 on balloon 1606. Next the distal balloon 1602, proximal balloon 1604 and medial balloon 1606 are immediately deflated and the catheter 1600 is removed. A second catheter 200 depicted in FIG. 2 is then advanced to the treatment site and stent 100 (FIG. 1) is then deployed. Alternatively, stent 100 can be deployed first using catheter 200 (FIG. 2) followed by administering the IkBα expressing vector using catheter 1600 as described above.

In another embodiment of the present invention, a CRM-1-binding compound-coated stent 100 is deployed to a treatment site using methods known to those skilled in the art of interventional cardiology and as described briefly herein. After the stent 100 has been placed, an injection catheter as depicted in FIG. 3 is advanced to the treatment site. The injection catheter has a C-shaped configuration comprising a catheter balloon 20 prior to inflation which has an injection needle 24 nested therein. Needle 24 has an injection port 26 that can be used to inject the IkBα expressing vector composition into the adventia from a proximal reservoir (not shown) located outside the patient.

After locating the injection catheter at the treatment site, the balloon 20 is inflated as depicted in FIG. 4. The inflated balloon 30 attached to the catheter body 28 and injection needle 24 capable of penetrating the adventitia. In FIG. 5 deployment of the IkBα expressing vector of the present invention is directly into the adventitia 34. The injection needle 24 penetrates the blood vessel wall 32 as balloon 20 is inflated and injects the IkBα expressing vector 36 into the tissue.

It is understood by those skilled in the art that the adventitial injection of the IkBα expressing vector of the present invention may be done before or after the deployment of the leptomycin B-eluting stent.

The following examples are provided to more precisely define and enable the medical devices and methods of the present invention. It is understood that there are numerous other embodiments and methods of using the present invention that will be apparent embodiments to those of ordinary skill in the art after having read and understood this specification and examples. Moreover, it is understood that the combination of leptomycin B and an IkBα-expressing vector are but one example of the compounds that can be used according to the teachings of the present invention. These alternate embodiments are considered part of the present invention.

### EXAMPLE 1

### In vitro Cell Culture Testing using Human Coronary Artery Smooth Muscle Cells

Leptomycin B was studied to evaluate its effect on human coronary artery smooth muscle cells (hCASMCs) and estimate its in vitro safety and efficacy profile. Leptomycin B was purchased from LC Laboratories. Wobum, Mass., USA catalogue number L-6100 and stored immediately upon arrival in a −40° C. freezer until used. The drug was purchased at a concentration of 54 μg/mL in ethanol.

Leptomycin B's anti-proliferative efficacy was tested at different concentrations (0.01 nM, 0.1 nM, 1.0 nM, 10.0 nM, 100 nM and 1000 nM) using a three day exposure to hCASMC. The same experiment was performed twice at two separate times to ascertain leptomycin's effect on cell proliferation using a cell viability assay, and the cell phenotype through a qualitative observation of cell morphology following the 3-day exposure to leptomycin B.

Proliferation Assay Protocol

The CellTiter-Glo® Luminescent Cell Viability Assay (Promega Corporation, Madison, Wis.) is a method of determining the number of viable cells in culture based on the quantitation of ATP, as an indicator of metabolically active cells (proliferating). A single reagent is added to cells directly in culture, this reagent lyses the cells and provides the luciferase enzyme that reacts with the liberated ATP to create a luminescent signal.

Day 0:

Twenty-four well plates were seeded with hCASMC at 5×10³ cells per well (n=3) using methods know to those skilled in the art of cell culture. For example and not intended as a limitation: Materials
1. Human coronary smooth muscles cells (HCASMC) are obtained from Clonetics, a division of Cambrex, Inc.
2. HCASMC basal media, supplied by Clonetics and supplemented with fetal bovine serum, insulin, hFGF-B (human fibroblast growth factor) hEGF (human epidermal growth factor).
3. Leptomycin B was purchased from LC Laboratories. Wobum, Mass., USA catalogue number L-6100
4. Absolute methanol
5. Twenty-four well polystyrene tissue culture plates

Day 1:

Dilutions of the drug were made in serum-free cell culture media from the stock solution. A volume of 100 μL of each dilution was added to designated wells. A volume of 100 μL of fresh culture media was added to control wells. The final volume per well was 1 mL in a concentration of 0.01 nM, 0.1 nM, 1.0 nM, 10.0 nM, 100 nM and 1000 nM, each drug concentration was tested in triplicate. Controls included wells with cells and without drug.

Day 3:

Cell Viability

The CellTiter-Glo® Luminescent Cell Viability Assay (Promega Corporation, Madison, Wis.) was performed to assess percent viability. The CellTiter-Glo® Luminescent Cell Viability Assay is a homogeneous method of determining the number of viable cells in culture based on quantitation of the ATP present, which signals the presence of metabolically active cells. The CelITiter-Glo® Assay is designed for use with multiwell plate formats cell proliferation and cytotoxicity assays. The homogeneous assay procedure involves addition of a single reagent (CelITiter-Glo® Reagent) directly to cells cultured in serum-supplemented medium. Cell washing, removal of medium or multiple pipetting steps are not required.

The homogeneous “add-mix-measure” format results in cell lysis and generation of a luminescent signal proportional to the amount of ATP present. The amount of ATP is directly proportional to the number of cells present in culture in agreement with previous reports. The CellTiter-Glo® Assay generates a “glow-type” luminescent signal, produced by the luciferase reaction, which has a half-life of greater than five hours.

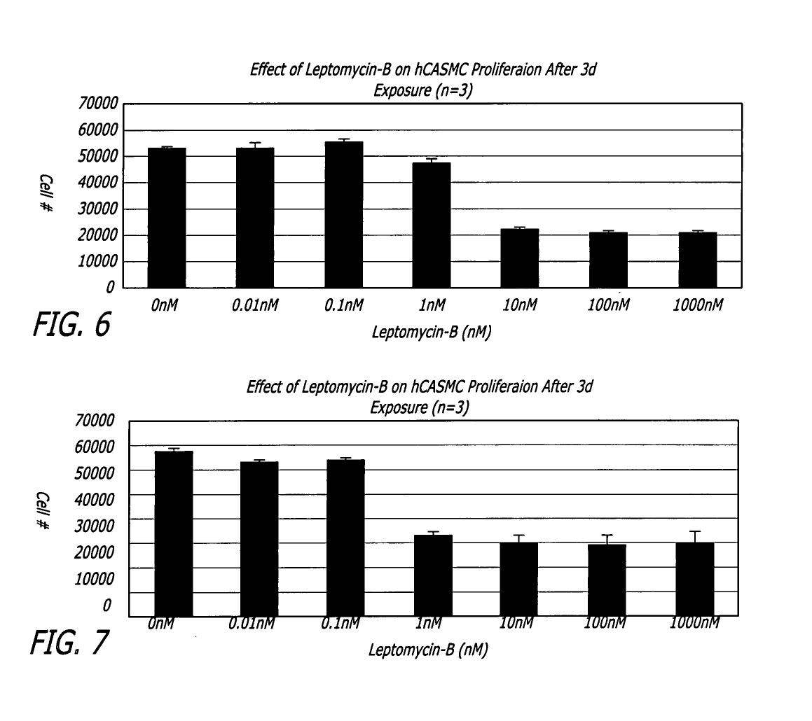

The proliferation assay and the microscopic evaluation showed that leptomycin B has an inhibitory effect on human coronary artery smooth muscle cell proliferation. The results are shown in FIGS. 6 and 7, corresponding to proliferation assay results from two experiments performed at separate times

Microscopic Evaluation of Cell Morphology

The evaluation of cell phenotype on drug-treated wells was performed by optical microscopy observation and capture of representative images at 200× (total magnification). Microscopic evaluation and pictures were performed before the proliferation assay was done on the 24-well drug-treated plate.

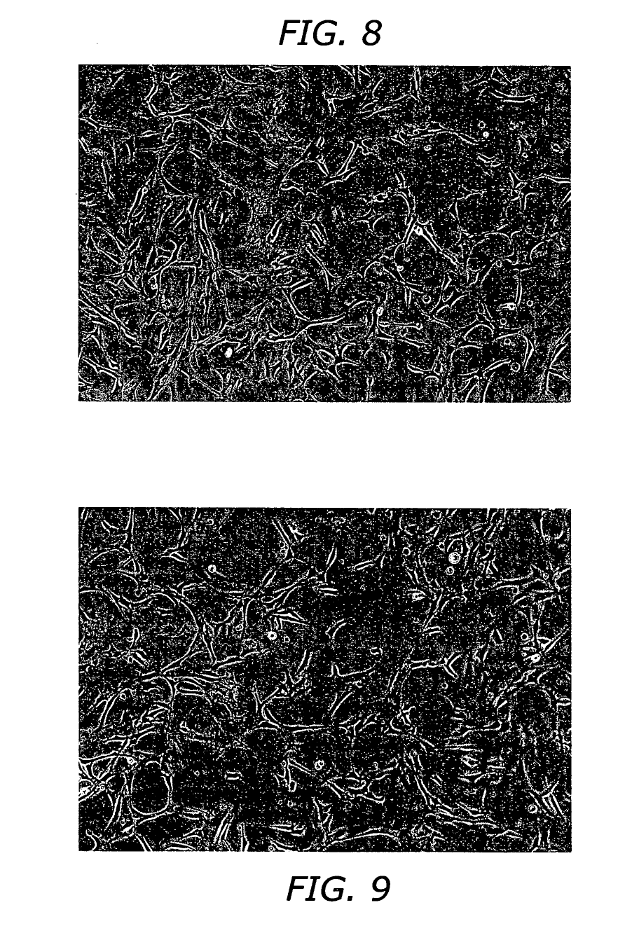

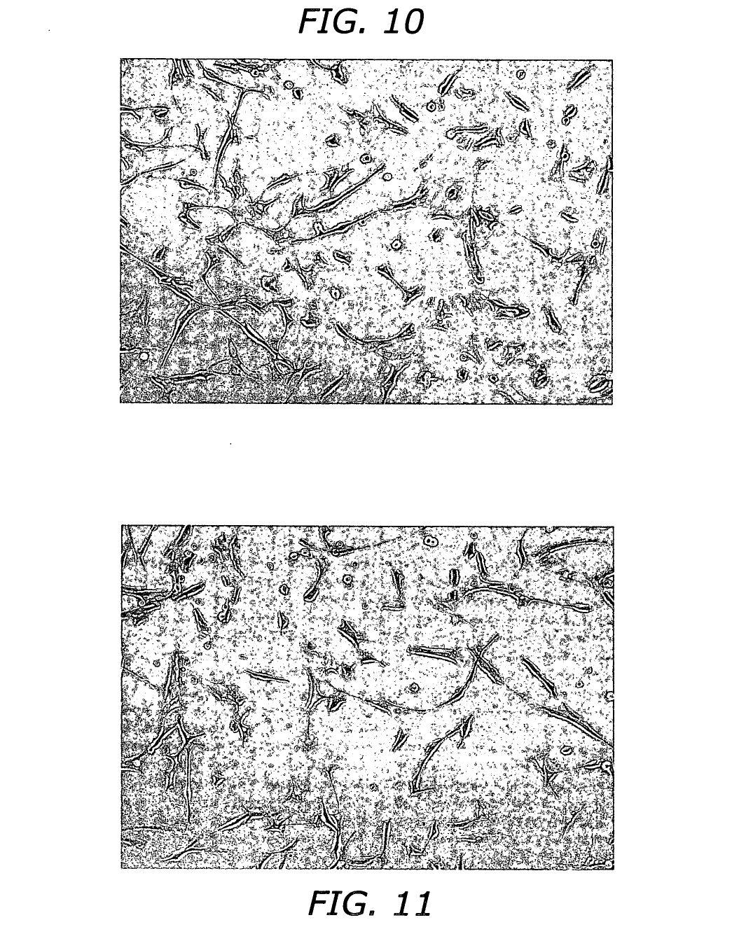

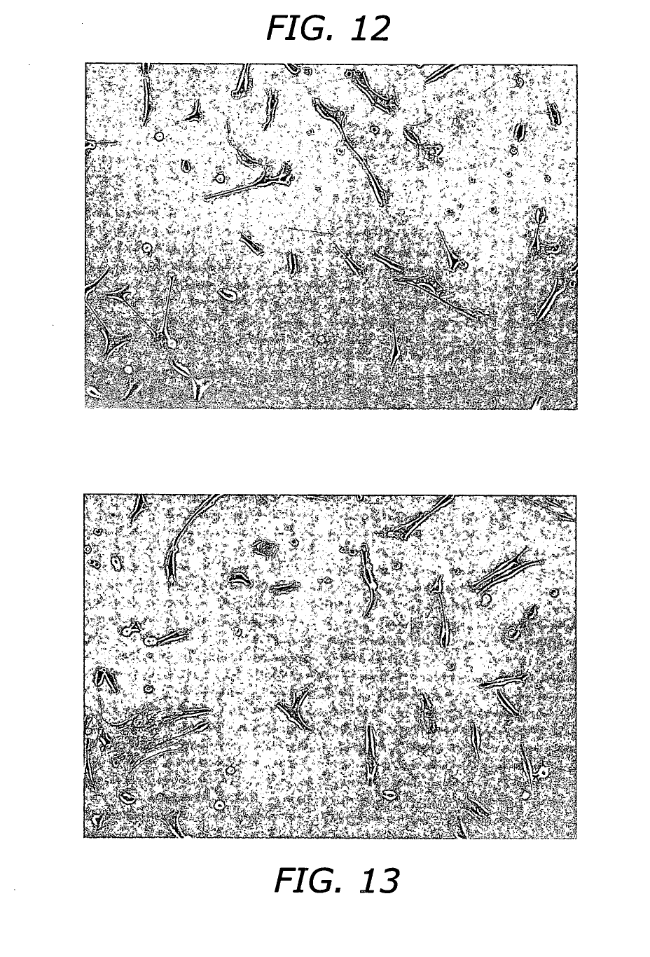

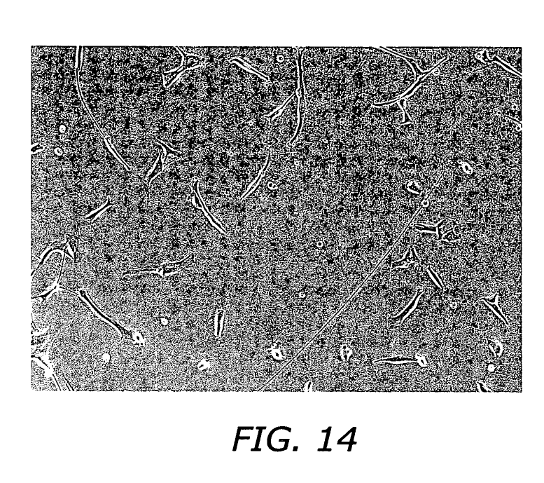

Representative microphotographs (See FIGS. 8-14) of cells exposed to different concentrations of leptomycin B for a period of three days. HCASMCs cultured in the absence of the drug (FIG. 8) reached a ˜90% confluency with the expected healthy phenotype at three days (duration of the assay), providing an adequate control. Cells cultured in the presence of leptomycin B had a decreased level of confluence. No microscopic signs of cytotoxicity were evidenced along the range of concentration evaluated.

These experiments demonstrate that leptomycin B has an inhibitory effect on human coronary artery smooth muscle cell proliferation in vitro at concentrations as low as 1 nM, and that the effect appears to be predominantly cytostatic rather than cytotoxic. Because smooth muscle cell hyperproliferation is the hallmark characteristic of restenosis, these results indicate that leptomycin B is useful for treating and inhibiting restenosis in vivo.

### EXAMPLE 2

### In vivo Testing of a Leptomycin B-coated Vascular Stent in a Porcine Model

Stent Preparation

Stainless steel stents are placed a glass beaker and covered with reagent grade or better hexane. The beaker containing the hexane immersed stents is then placed into an ultrasonic water bath and treated for 15 minutes at a frequency of between approximately 25 to 50 KHz. Next the stents are removed from the hexane and the hexane was discarded. The stents are then immersed in reagent grade or better 2-propanol and the vessel containing the stents and the 2-propanol is treated in an ultrasonic water bath as before. Following cleaning the stents with organic solvents, they are thoroughly washed with distilled water and thereafter immersed in 1.0 N sodium hydroxide solution and treated at in an ultrasonic water bath as before. Finally, the stents are removed from the sodium hydroxide, thoroughly rinsed in distilled water and then dried in a vacuum oven over night at 40° C. After cooling, the clean-dried stents are provided with a leptomycin B-eluting coating comprising a polymer primer coat and a drug-Matrix polymer coating using methods know to those skilled in the art together with the teachings provided herein and the incorporated references.

Experimental Design

The swine has emerged as the most appropriate animal model for the study of the endovascular devices. The anatomy and size of porcine coronary vessels are comparable to that of humans. Furthermore, the neointimal hyperplasia that occurs in response to vascular injury is similar to that seen clinically in humans. Results obtained in the swine animal model are considered predictive of clinical outcomes in humans. Consequently, regulatory agencies have deemed six-month data in the porcine sufficient to allow progression to human trials. Therefore, as used herein “animal” shall include mammals, fish, reptiles and birds. Mammals include, but are not limited to, primates, including humans, dogs, cats, goats, sheep, rabbits, pigs, horses and cows.

The ability to reduce neointimal hyperplasia in response to intravascular stent placement in an acutely injured porcine coronary artery is demonstrated in the following example. Two controls and three treatment arms are used as outlined below:

Control Groups: Six animals are used in each control group. The first control group tests the anti-restenotic effects of the clean, dried MedtronicAVE S7 stents having neither polymer nor drug coatings. The second control group tests the anti-restenotic effects of polymer alone. Clean, dried MedtronicAVE S7 stents having Matrix polymer coatings without drug are used in the second control group.

Experimental Treatment Groups: Twelve animals are included in each group. MedtronicAVE S7 stents having a coating comprising a Matrix polymer coating containing 35% leptomycin B by weight in accordance with the teachings of the present invention are used.

Non-atherosclerotic acutely injured right carotid artery (RCA), left anterior descending (LAD), and/or left circumflex (LCX) arteries of the Farm Swine (or miniswine) are utilized in this study. Placement of coated and control stents is random by animal and by artery. The animals are handled and maintained in accordance with the requirements of the Laboratory Animal Welfare Act (P.L. 89-544) and its 1970 (P.L. 91-579), 1976 (P.L. 94-279), and 1985 (P.L. 99-198) amendments. Compliance is accomplished by conforming to the standards in the Guide for the Care and the Use of Laboratory Animals, ILAR, National Academy Press, revised 1996. A veterinarian performs a physical examination on each animal during the pre-test period to ensure that only healthy pigs are used in this study.

A. Pre-Operative Procedures

The animals are monitored and observed 3 to 5 days prior to experimental use. The animals had their weight estimated at least 3 days prior to the procedure in order to provide appropriate drug dose adjustments for body weight. At least one day before stent placement, 650 mg of aspirin is administered. Animals are fasted twelve hours prior to the procedure.

B. Anesthesia

Anesthesia is induced in the animal using intramuscular Telazol and Xylazine. Atropine is administered (20 μg/kg I.M.) to control respiratory and salivary secretions. Upon induction of light anesthesia, the subject animal is intubated. Isoflurane (0.1 to 5.0% to effect by inhalation) in oxygen is administered to maintain a surgical plane of anesthesia. Continuous electrocardiographic (ECG) monitoring is performed. An I.V. catheter is placed in the ear vein in case it is necessary to replace lost blood volume. The level of anesthesia is monitored continuously by ECG and the animal's response to stimuli.

C. Catheterization and Stent Placement

Following induction of anesthesia, the surgical access site is shaved and scrubbed with chlorohexidine soap. An incision is made in the region of the right or left femoral (or carotid) artery and betadine solution is applied to the surgical site. An arterial sheath is introduced via an arterial stick or cutdown and the sheath is advanced into the artery. A guiding-catheter is placed into the sheath and advanced via a 0.035″ guide wire as needed under fluoroscopic guidance into the ostium of the coronary arteries. An arterial blood sample is obtained for baseline blood gas, ACT and HCT. Heparin (200 units/kg) is administered as needed to achieve and maintain ACT ≧300 seconds. Arterial blood pressure, heart rate, and ECG are recorded.

After placement of the guide catheter into the ostium of the appropriate coronary artery, angiographic images of the vessels are obtained in at least two orthagonal views to identify the proper location for the deployment site. Quantitative coronary angiography (QCA) is performed and recorded. Nitroglycerin (200 μg I.C.) may be administered prior to treatment and as needed to control arterial vasospasm. The delivery system is prepped by aspirating the balloon with negative pressure for five seconds and by flushing the guidewire lumen with heparinized saline solution.

Deployment, patency and positioning of stent are assessed by angiography and a TIMI (Thrombolysis In Myocardial Infarction) score is recorded. Results are recorded on video and cine. Final lumen dimensions are measured with QCA and/or intravascular ultrasound (IVUS). These procedures are repeated until a device is implanted in each of the three major coronary arteries of the pig. The stents are deployed having an expansion ratio of 1:1.2. After final implant, the animal is allowed to recover from anesthesia. Aspirin is administered at 325 mg orally daily until sacrificed 28 days later.

D. Follow-Up Procedures and Termination

After 28 days, the animals are anesthetized and a 6F arterial sheath is introduced and advanced. A 6F large lumen guiding-catheter (diagnostic guide) is placed into the sheath and advanced over a guide wire under fluoroscopic guidance into the coronary arteries. After placement of the guide catheter into the appropriate coronary ostium, angiographic images of the vessel are taken to evaluate the stented sites. At the end of the re-look procedure, the animals are euthanized with an overdose of pentabarbitol I.V. and KCI I.V. The heart, kidneys, and liver are harvested and visually examined for any external or internal trauma. The organs are flushed with 1000 mL of lactated ringers at 100 mmHg and then flushed with 1000 mL of formalin at 100-120 mmHg. All organs are stored in labeled containers of formalin solution.

E. Histology and Pathology

The stented vessels are X-rayed prior to histology processing. The stented segments are processed for routine histology, sectioned, and stained following standard histology lab protocols. Appropriate stains are applied in alternate fashion on serial sections through the length of the treated vessels.

F. Data Analysis and Statistics

1. QCA Measurement

Quantitative angiography is performed to measure the balloon size at peak inflation as well as vessel diameter pre- and post-stent placement and at the 28 day follow-up. The following data are measured or calculated from angiographic data:

Stent-to-artery-ratio

Minimum lumen diameter (MLD)

Distal and proximal reference lumen diameter

Percent Stenosis=(Minimum lumen diameter÷reference lumendiameter)×100

2. Histomorphometric Analysis

Histologic measurements are made from sections from the native proximal and distal vessel and proximal, middle, and distal portions of the stent. A vessel injury score is calculated using the method described by Schwartz et al. (Schwartz RS et al. Restenosis and the proportional neointimal response to coronary artery injury: results in a porcine model. J Am Coll Cardiol 1992; 19:267-74). The mean injury score for each arterial segment is calculated. Investigators scoring arterial segment and performing histopathology are “blinded” to the device type. The following measurements are determined:

a) External elastic lamina (EEL) area

b) Internal elastic lamina (IEL) area

c) Luminal area

d) Adventitial area

e) Mean neointimal thickness

f) Mean injury score

3. The neointimal area and the % of in-stent restenosis are calculated as follows:

a) Neointimal area=(IEL−luminal area)

b) In-stent restenosis=[1−(luminal area÷IEL)]×100.

A given treatment arm is deemed beneficial if treatment results in a significant reduction in neointimal area and/or in-stent restenosis compared to both the bare stent control and the polymer-on control.

G. Surgical Supplies and Equipment

The following surgical supplies and equipment are required for the procedures described above:
a) Standard vascular access surgical tray
b) Non-ionic contrast solution
c) ACT machine and accessories
d) HCT machine and accessories (if applicable)
f) Respiratory and hemodynamic monitoring system
g) IPPB Ventilator, associated breathing circuits and Gas Anesthesia Machine
h) Blood gas analysis equipment
i) 0.035″ HTF or Wholey modified J guidewire, 0.014″ Guidewires
j) 6, 7, 8, and 9F introducer sheaths and guiding catheters (as applicable)
k) Cineangiography equipment with QCA capabilities
l) Ambulatory defibrillator
m) Standard angioplasty equipment and accessories
n) IVUS equipment (if applicable)
o) For radioactive labeled cell studies (if applicable):
p) Centrifuge
q) Aggregometer
r) Indium 111 oxime or other as specified
s) Automated Platelet Counter
d) Radiation Detection Device

F. Results

Medtronic S7 stents (18 mm×3-3.5 mm diameter) are coated as described herein and sterilized and implanted into farm swine at an expansion ratio of 1:1.2 as described above. Animals are allowed to recover, and held for 28 d, after which the animal is euthanized and the tissue fixed and processed for histochemistry and histomorphometry, using standard techniques. The neointimal thickness and injury score are measured at proximal and distal stent segments. A good correlation is observed between the injury score and neointimal thickness in the bare stent control group. A significant decrease in the neointimal thickness when the injury score increases are observed when the data from the treatment groups versus the bare stent controls.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A medical device for providing the controlled-release of an anti-restenotic composition comprising:
a vascular stent having a generally cylindrical shape comprising an outer surface, an inner surface, a first open end, a second open end and wherein at least one of said inner or said outer surfaces are adapted to provide the controlled-release of an anti-restenotic effective amount of at least one I kappa B alpha (IkBα)nuclear factor kβ (NFkβ) complex breakdown inhibitor.

2. The medical device according to claim 1 wherein said stent is mechanically expandable.

3. The medical device according to claim 1 wherein said stent is self expandable.

4. The medical device according to claim 1 wherein said at least one IkBα-NFkβ complex breakdown inhibitor is present on both said inner surface and said outer surface of said stent.

5. The medical device according to claim 1 wherein at least one of said inner or said outer surfaces are coated with a polymer wherein said polymer has at least one IkBα-NFkβ complex breakdown inhibitor incorporated therein and said polymer releases said at least IkBα-NFkβ complex breakdown inhibitor into a tissue of a mammal.

6. The medical device according to claim 1 wherein said at least one IkBα-NFkβ complex breakdown inhibitor inhibits or interferes with the normal biological function of a CRM-1 (chromosome region maintenance-1).

7. The medical device according to claim 6 wherein said at least one IkBα-NFkβ complex breakdown inhibitor is leptomycin B and derivatives and analogues thereof.

8. The medical device according to claim 1 wherein said stent is delivered to said tissue of an anatomical lumen of a mammal using a balloon catheter.

9. The medical device according to claim 8 wherein said tissue is a blood vessel lumen.

10. The medical device according to claim 5 wherein said polymer is selected from the group consisting of polyurethanes, silicones, polyolefins, polyisobutylene, ethylene-alphaolefin copolymers, acrylic polymers and copolymers, ethylene-co-vinylacetate, polybutylmethacrylate, vinyl halide polymers and copolymers, polyvinyl chloride; polyvinyl ethers, polyvinyl methyl ether, polyvinylidene halides, polyvinylidene fluoride, polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate; cellophane, cellulose nitrate, cellulose propionate, cellulose ethers, carboxymethyl cellulose and combinations thereof.

11. A vascular stent comprising a polymeric coating containing an anti-restenotic effective amount of at least one IkBα-NFkβ complex breakdown inhibitor.

12. The vascular stent of claim 11 further comprising a parylene primer coat.

13. The vascular stent of claim 11 wherein said polymeric coating comprises a terpolymer-copolymer-homopolymer blend.

14. The vascular stent according to claim 13 wherein said terpolymer-copolymer-homopolymer blend comprises approximately 60% to 70% terpolymer, approximately 20% to 25% copolymer and approximately 5% to 15% homopolymer.

15. The vascular stent according to claim 14 wherein said terpolymer comprises from approximately 70% to 80% hexyl methacrylate, approximately 1%-10% vinyl acetate and approximately 15% to 20% polyvinylpyrrolidone (PVP); the copolymer comprises from approximately 90% to 99% butyl methacrylate and approximately from 1% to 10% vinyl acetate; and the homopolymer is PVP.

16. The vascular stent according to claim 13 wherein said terpolymer-copolymer-homopolymer blend comprises approximately 67% of a terpolymer having 77% hexyl methacrylate, 5% vinyl acetate and 18% PVP; approximately 23% of a copolymer comprising 95% butyl methacrylate and 5% vinyl acetate and approximately 10% of the homopolymer PVP.

17. The vascular stent of claim 1 or claim 11 wherein said at least one IkBα-NFkβ complex breakdown inhibitor is in a concentration of between approximately 0.001 % to 99% by weight of IkBα-NFkβ complex breakdown inhibitor-to-polymer.

18. The vascular stent of claim 1 or claim 11 wherein said at least one IkBα-NFkβ complex breakdown inhibitor is a CRM-1 protein binding compound.

19. The vascular stent according to claim 18 wherein said CRM-1 protein binding compound is leptomycin B.

20. The vascular stent of claim 18 wherein said at least one IkBα-NFkβ complex breakdown inhibitor comprises a CRM-1 protein binding compound and a nucleic acid encoding for mammalian IkBα.

21. The vascular stent of claim 20,wherein said nucleic acid encoding for mammalian IkBα further comprises a replication-defective viral vector.

22. The vascular stent according to claim 21 wherein said replication-defective viral vector is selected from the group consisting of adenoviruses, retroviruses, lentiviruses, alphaviruses, and herpesviruses.

23. The vascular stent according to claim 22 wherein said replication-defective viral vector is an adenovirus.

24. The vascular stent according to claim 22 wherein said replication-defective viral vector is an alphavirus.

25. The vascular stent according to claim 22 wherein said replication-defective viral vector is a retrovirus.

26. The vascular stent according to claim 22 wherein said replication-defective viral vector is a lentivirus.

27. The vascular stent according to claim 22 wherein said replication-defective viral vector is a herpesvirus.

28. The vascular stent of claim 20 wherein said nucleic acid encoding for mammalian IkBα further comprises a liposome.

29. The vascular stent according to claim 11 wherein said stent is delivered to a tissue of a mammal's anatomical lumen using a balloon catheter.

30. The vascular stent of claim 20 wherein said a CRM-1 protein binding compound and nucleic acid encoding for mammalian IkBα act synergistically to inhibit restenosis.

31. A method for treating or inhibiting restenosis comprising administering to a treatment site a vascular stent having a coating comprising at least one IkBα-NFkβ complex breakdown inhibitor.

32. The method for treating or inhibiting restenosis according to claim 31 wherein said IkBα-NFkβ complex breakdown inhibitor comprises a CRM-1 protein binding compound.

33. The method for treating or inhibiting restenosis according to claim 32 wherein said CRM-1 protein binding compound is leptomycin B.

34. A method for treating or inhibiting restenosis comprising administering to a treatment site at least two IkBα-NFkβ complex breakdown inhibitors wherein a first IkBα-NFkβ complex breakdown inhibitor comprises a CRM-1 protein binding compound and a second IkBα-NFkβ complex breakdown inhibitor comprises nucleic acid encoding for mammalian IkBα.

35. The method for treating or inhibiting restenosis according to claim 34 wherein said first and said second IkBα-NFkβ complex breakdown inhibitors are administered to a treatment site form the same vascular stent.

36. The method for treating or inhibiting restenosis according to claim 34 wherein said first IkBα-NFkβ complex breakdown inhibitor is administered to a treatment site using a vascular stent and said second IkBα-NFkβ complex breakdown inhibitor is administered to a treatment site using a catheter.

37. The method for treating or inhibiting restenosis according to claim 36 wherein said first IkBα-NFkβ complex breakdown inhibitor is administered to a treatment site using a vascular stent and said second IkBα-NFkβ complex breakdown inhibitor is administered to a treatment site using a catheter to deliver said second IkBα-NFkβ complex breakdown inhibitor to the luminal lining of a vessel.

38. The method for treating or inhibiting restenosis according to claim 36 wherein said first IkBα-NFkβ complex breakdown inhibitor is administered to a treatment site using a vascular stent and said second IkBα-NFkβ complex breakdown inhibitor is administered to a treatment site using a catheter to deliver said second IkBα-NFkβ complex breakdown inhibitor to the adventitia of a vessel.

39. The method for treating or inhibiting restenosis according to any one of claims 31 to 38 wherein said CRM-1 protein binding compound is leptomycin B and derivatives and analogues thereof.

40. A vascular stent consisting essentially of a controlled-release coating and leptomycin B.

41. The vascular stent according to claim 40 wherein said controlled-release coating comprises a polymeric primer coat and a polymeric drug-releasing polymer blend.
